(19) United States
(12) Patent Application Publication
(10) Pub. No.: US 2025/0275690 A1
(43) Pub. Date: Sep. 4, 2025

(54) METHODS AND SYSTEMS FOR CAPTURING MUSCLE ACTIVITY USING PIEZOELECTRIC TRANSDUCERS

(71) Applicant: deltaPress Solutions Incorporated, Houston, TX (US)

(72) Inventors: Anirudh Prasanna Srinivasan, Houston, TX (US); Jayanth Sairam Pratap, Waltham, MA (US)

(21) Appl. No.: 19/070,025

(22) Filed: Mar. 4, 2025

Related U.S. Application Data
(60) Provisional application No. 63/561,094, filed on Mar. 4, 2024.

(51) Int. Cl. A61B 5/11; A61B 5/00

(52) U.S. Cl. CPC A61B 5/1126; A61B 5/1123; A61B 5/6802; A61B 5/7225; A61B 5/7264

(57) ABSTRACT

The disclosed system includes a wearable device with piezoelectric transducers for capturing muscle activity by measuring dynamic surface pressure differentials created by muscle contractions. The device includes multiple sensors at points of muscular activity. The hardware component, comprising the transducers, circuitry, and a microprocessor, generates an analog signal proportional to the pressure applied, which is then converted into a digital signal. The software component utilizes a machine learning model to interpret the data in real time for gesture recognition and activity monitoring. The data collection method is an analog process with no associated scaling power cost, allowing for high sensor density and accurate capture of activity.

### CROSS-REFERENCE TO RELATED APPLICATIONS

This application claims priority to U.S. Provisional Application No. 63/561,094 filed Mar. 4, 2024, which is incorporated by reference in its entirety.

### FIELD OF THE DISCLOSURE

The present disclosure generally relates to the field of wearable technology and sensor systems, and more specifically, to methods and systems for capturing muscle activity using piezoelectric elements to measure dynamic surface pressure differentials and categorize movements based on these differentials.

### BACKGROUND

Conventional technology for capturing and analyzing human movement and muscle activity includes methods such as electromyography (EMG), which measures the electrical activity produced by skeletal muscles, and its non-invasive variant, surface electromyography (sEMG), which involves placing electrodes on the skin. Other conventional methods such as subcutaneous electromyography requires inserting needles into the muscle, while vibromyography (VMG) quantifies muscular activity by measuring vibrations or mechanical energy during muscular contractions using microelectromechanical systems. These methods, however, face challenges in scalability, are often burdened by noise and external manipulation, and may require invasive procedures or power-consuming devices that limit their practicality and ease of use in various applications. These and other deficiencies exist. Thus, there is currently a need for systems that recognize muscle movements and are highly scalable without low power consumption.

### SUMMARY OF THE DISCLOSURE

In some embodiments, the techniques described herein relate to a system for capturing muscle activity, including: a wearable device including: one or more piezoelectric elements configured to convert one or more mechanical deformations associated with muscle activity into an analog signal; an analog-to-digital converter (ADC) electrically coupled to the piezoelectric elements, the ADC being configured to convert the analog signal into a digital signal; and a processor in communication with the ADC, wherein the processor is configured to receive and analyze the digital signal, wherein the processor is further configured to determine, based on the analysis, an action associated with the digital signal based on the analysis.

In some embodiments, the techniques described herein relate to a method for capturing muscle activity, including: providing a wearable device including one or more piezoelectric elements; positioning the one or more piezoelectric elements in proximity to a user's muscles; converting, by the piezoelectric elements, mechanical deformations associated with muscle activity into an analog signal; converting, by an analog-to-digital converter (ADC) electrically coupled to the piezoelectric elements, the analog signal into a digital signal; analyzing, by a processor in communication with the ADC, the digital signal; and determining, by the processor, an action associated with the digital signal based on the analysis.

In some embodiments, the techniques described herein relate to a non-transitory computer readable medium containing computer executable instructions that, when executed by a computer hardware arrangement, cause the computer hardware arrangement to perform procedures including: converting mechanical deformations associated with muscle activity into an analog signal; converting the analog signal into a digital signal; analyzing, by a machine learning algorithm, the digital signal; and determining an action associated with the digital signal based on the analysis.

Further features of the disclosed systems and methods, and the advantages offered thereby, are explained in greater detail hereinafter with reference to specific example embodiments illustrated in the accompanying drawings.

### BRIEF DESCRIPTION OF THE DRAWINGS

In order to facilitate a fuller understanding of the present embodiments, reference is now made to the attached drawings. The drawings should not be construed as limiting the present embodiments, but are intended only to illustrate different aspects and embodiments of the invention.

FIG. 1 is a block diagram illustrating a system according to example embodiments.

FIG. 2 is a diagram illustrating wearable elements according to example embodiments.

FIG. 3 is a block diagram illustrating a circuitry of a wearable element according to example embodiments.

FIG. 4 is a diagram illustrating a process according to example embodiments.

FIG. 5 is a flowchart illustrating a method according to example embodiments.

FIGS. 6A, 6B, and 6C illustrate two-dimensional representations of time series according to example embodiments.

FIG. 7 illustrate a multi-dimensional representation of time series according to example embodiments.

FIG. 8 is a block diagram illustrating a system according to example embodiments.

### DETAILED DESCRIPTION

Exemplary embodiments will now be described in order to illustrate various features of the invention. The embodiments described herein are not intended to be limiting as to the scope of the invention, but rather are intended to provide examples of the components, use, and operation of the invention.

The present disclosure relates to a system and method for capturing and interpreting muscle activity. The system may include a wearable device that uses piezoelectric transducers to measure dynamic surface pressure differentials created by muscle contractions on a user. The piezoelectric transducers may be strategically placed at points of muscular activity on the wearer. The wearable device may be designed to be worn on various parts of the body, depending on the specific application or the particular muscle groups that are of interest.

In some example embodiments, the system includes a hardware component comprising of a plurality of piezoelectric transducers, circuitry, and a microprocessor. The piezoelectric transducers may operate using the piezoelectric effect, generating an analog signal directly proportional to the pressure applied along the sensor's sensitive axis. This signal may be rectified by the circuitry and fed into a processor, which may convert the analog signal into a digital signal. This method of data collection may be an analog process with no associated scaling power cost, making the transducers very scalable. The transducer density may be restricted by spatial constraints, not by power costs, allowing for a high sensor density wearable device.

In addition to the hardware components, the system may include a software component that utilizes on-board time series analysis performed by the microprocessor. By measuring the digital signals collected by the piezoelectric transducers and recording the data spatially as a time series, a machine learning model may interpret the data in real time. This interpretation may be used for gesture recognition and activity monitoring, providing a high-fidelity digital output of human movement.

The system and method disclosed herein may address some of the technical problems present in the field of wearable technology and sensor systems. For instance, current methods of measuring and analyzing movement may be difficult to scale up, burdened by noise and external manipulation, or require intensive data processing and analysis. The disclosed system and method, on the other hand, may provide an on-the-go, inexpensive method that can successfully and accurately track the actions performed by the full human body. This may result in a more effective and efficient way of capturing and analyzing muscle activity, potentially improving human-machine interfaces, activity monitoring, and sports analytics. In addition, the system has high scalability due to zero power consumption from the piezoelectric sensors. This method of capturing muscle activity may be beneficial in various fields such as health technology, sports, and augmented/virtual/extended reality (AR/VR/XR).

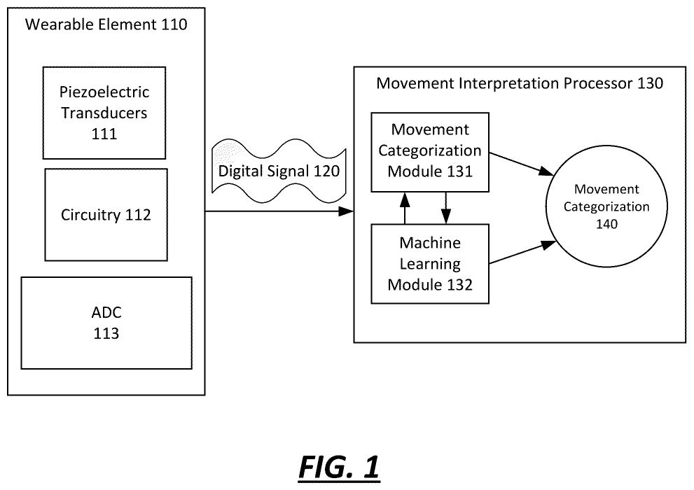

FIG. 1 is a block diagram illustrating a system 100 according to exemplary embodiments. The system 100 may include a wearable element 110 and a movement interpretation processor 130. The wearable element 110 can include one or more piezoelectric transducers 111, circuitry 112, and an analog to digital converter (ADC) 113 connected to the piezoelectric transducers 111 by the circuitry 112. The movement interpretation processor 130 can include a movement categorization module 131 and machine learning module 132. These modules may be collections of code or instructions stored on a media that represent a series of machine instructions that implement one or more actions explained below. Each of these modules can be stored within a memory or data storage unit associated with the wearable element 110.

The wearable element 110 may include a strap, patch, band, glove, headband, sleeve, or other item that can be worn or removably attached to a human user. The wearable element 110 could also take the form of a belt, anklet, knee brace, elbow brace, shoulder brace, chest strap, or a full-body suit. It could also be integrated into clothing items such as shirts, pants, socks, shoes, or hats. In some embodiments, the wearable element could be a skin patch that adheres directly to the skin. Furthermore, the wearable element could be designed as a piece of jewelry such as a bracelet, necklace, or ring. In other embodiments, the wearable element 110 could be a component of a prosthetic or orthotic device. In other embodiments, the wearable element 110 may comprise an element that can be held, grasped, or grabbed by the human user. Examples of such elements could include a handheld device, similar to a joystick or game controller, that is equipped with piezoelectric transducers. The user's grip and hand movements could then be monitored and analyzed. A stylus or pen-like device, which could be used to capture fine motor movements of the hand and fingers, such as those used in writing or drawing. In other embodiments, a ball or sphere that can be squeezed or manipulated by the user. This could be particularly useful in physical therapy or rehabilitation settings, where grip strength and hand flexibility are often areas of focus. This could allow for the capture of complex hand and finger movements, such as those used in sign language or in playing musical instruments.

In some embodiments, the wearable element 110 can be removably attached to the user using any suitable means on the arms, legs, torso, head, or other body parts. As a nonlimiting example, the wearable element can include a strap, Velcro, buttons, adhesives, or simply be held by the hands of the user. The wearable element can be mad of any suitable material such as plastic, cloth, leather, neoprene, silicone, elastomer, nylon, polyester, spandex, or a combination of these materials. It could also be made from advanced materials like smart textiles or e-textiles, which are fabrics that enable digital components and electronics to be embedded in them. Furthermore, the material could be breathable, waterproof, or sweat-resistant to enhance user comfort and device durability.

The wearable element 110 can include a plurality of piezoelectric transducers 111. These elements may include materials that produce the piezoelectric effect. Generally, piezoelectric materials are substances that generate an electric charge in response to mechanical stress or mechanical deformation such as muscle flexion or movement. When pressure or any kind of mechanical force is applied to these piezoelectric materials, they produce a voltage or analog signal. In some embodiments, the piezoelectric transducers 111 may generate an electric signal proportional to the force applied on it. In other example embodiments, the piezoelectric transducers 111 can include one or more piezoelectric crystals or other materials suitable for conducting piezoelectric energy. The number of piezoelectric transducers 111 can include a wide range, including without limitation a few elements to several dozen elements per wearable element 110.

The one or more piezoelectric transducers 111 can be placed on a person's body, e.g. their ankle or their wrist. As a result of the force acting on the piezoelectric transducers 111, the piezoelectric transducers 111 will produce an analog signal. The analog signal may be directly proportional to the pressure applied along the piezoelectric transducers' 111 sensitive axis. In some embodiments, because the signal is generated through an analog process, there is no input power required to generate these analog signals, thereby providing immense scalability of the piezoelectric transducers 111. These analog signals can travel through the circuitry 112 and into the ADC 113. The ADC may convert the analog signal from the piezoelectric transducers 111 into a digital signal 120.

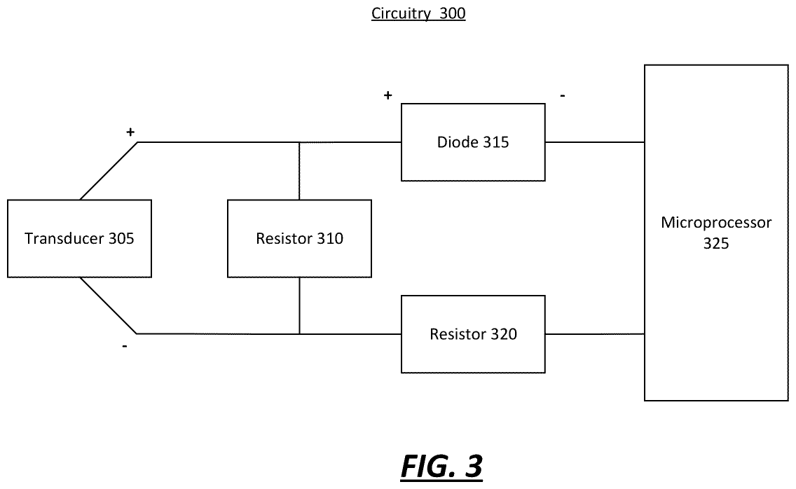

The circuitry 112 may include any suitable circuitry for transmitting the analog signal produced by the piezoelectric transducers 111 and sending them to the ADC 113 with further reference to the example embodiments in FIG. 3. The ADC 113 can comprise any suitable means for converting an analog signal into a digital signal 120, including without limitation successive approximation register (SAR) ADC, flash ADC, delta-sigma (δσ) ADC, pipelined ADC, and dual slope ADC. In some embodiments, the wearable element 110 can include many piezoelectric transducers connected to several ADCs 113 via several groups of circuitries 112.

In some embodiments, the wearable element 110 does not include any power source such as a battery or other wired or wireless power source. Instead, the ADC 113 and movement interpretation processor 130 are powered exclusively by the electric energy produced by the piezoelectric transducers 111. In such embodiments, the wearable element 110 reduces power needs and reduces the weight of the wearable element 110, thus giving the wearer greater freedom to move without restriction. Furthermore, since the digital signal 120 is not kinematic in nature, the digital signal 120 is significantly less prone to external noise and manipulation and can accurately capture activity on a 1-to-1 scale without drift. In addition, the high density of the piezoelectric transducers 111 as well as low-noise data allows for very precise translation of human movement into high-fidelity digital output. In other embodiments, the wearable element 110 may include a separate power source or be electrically connected to a power source. These power sources may include without limitation a battery, solar panel, or wired power sources such that the wearable element 110 may be plugged into a power outlet.

The wearable element 110 may transmit the digital signal 120 from the ADC 113 to the movement interpretation processor 130. The movement interpretation processor 130 may include a movement categorization module 131 and machine learning module 132. In some embodiments, the movement interpretation processor 130 may be powered by the energy generated by the piezoelectric transducers 111 when they are acted upon by the movement and flexion of the wearer. In other embodiments, the movement interpretation processor 130 may be powered by a separated power unit such as a battery, wired power unit, or wireless power unit. In still other embodiments, the movement interpretation processor 130 may be within the wearable element 110. Alternatively, the movement interpretation processor 130 may be separate from the wearable element 110 such that the digital signal 120 is sent over a wired or wireless network from the ADC 113 to the movement interpretation processor 130.

Either or both the movement categorization module 131 and the machine learning module 132 may record the digital signal 120 spatially as a time series. In other words, they capture the sequence of data points each representing the digital signal 120 from the piezoelectric transducers 111 at a specific point in time. The time series data is spatially recorded, indicating that the data points are not just chronologically ordered, but also associated with the specific spatial location of the piezoelectric transducers 111 on the wearable element 110. This spatial aspect of the time series data allows the system to understand not just when but also where on the body the muscle activity is occurring.

The movement categorization module 131 may directly receive the digital signal 120, while in other embodiments the machine learning module 131 may directly receive the digital signal 120. For example, the movement categorization module 131 may receive the digital signal 120 and interpret the digital signal 120 by inferring what the user is doing with their wearable element 110. For example, a person may be wearing the wearable element 110 around their wrist. When the person throws a ball, the area around the person's wrist experiences flexion, stretching, compression, and other movements during the throwing motion. In response, the piezoelectric transducers 111 generate analog signals based on this flexion, and the movement categorization module 131 interprets the corresponding digital signal 120. Based on the interpretation of the digital signal 120, the movement categorization module 131 may generate the movement categorization 140. The movement categorization module 131 can generate the movement categorization 140 as one or more data points. In an example embodiment, the movement categorization module 131 module compares the incoming digital signal 120 against a database or memory containing a plurality of signal patterns that have been previously identified and correlated with specific movements or gestures. The movement categorization module 131 can match the characteristics of the received digital signal 120, such as amplitude, frequency, and duration, to known patterns within the database. Once a match is found, the movement categorization module 131 categorizes the current movement by associating it with the corresponding action from the database, thereby generating a movement categorization 140.

In some embodiments, the digital signal 120 may be sent to the machine learning module 132 instead of or in addition to the movement categorization module 131. The machine learning module 132 may feed the digital signal 120 into a machine learning algorithm that is configured to interpret the digital signal 120 and determine the movement categorization 140 of the action being performed by the user associated with the wearable element 110. By utilizing pattern recognition algorithms, the machine learning module 132 can match the characteristics of the received signal, such as amplitude, frequency, and duration, to known patterns within the database. Once a match is found, the machine learning module 132 categorizes the current movement by associating it with the corresponding action from the database. In other example embodiments, the machine learning module 132 may decompose the time series data into its constituent components, such as trend, seasonality, and noise. Then, the machine learning module 132 may extract features from the time series data that are indicative of specific movements or gestures. These features could include statistical measures such as mean, variance, and skewness, as well as more complex features like Fourier transforms or wavelet coefficients that capture the frequency content of the signal. The movement categorization module 131 may use pattern recognition algorithms to compare the extracted features against a pre-defined set of patterns associated with known movements. By identifying the closest matching pattern, the movement categorization module 131 can categorize the current movement. Furthermore, the movement categorization module 131 may analyze the temporal dynamics of the time series data to understand how the signal evolves over time. This analysis may involve determining the degree of similarity between observations as a function of the time lag between them. In some embodiments, the modules may generate visualizations of the time series data and the results of the analysis to help interpret the findings. This could include line charts, heat maps, or other graphical representations that make the patterns in the data more apparent.

The machine learning module 132 may include one or more different kinds of machine learning models, including without limitation: supervised learning models such as linear regression models; logistic regression models; support vector machines (SVM), and neural networks such as convolutional neural networks (CNNs) and recurrent neural networks (RNNs); unsupervised learning models including clustering algorithms such as K-means and hierarchical clustering algorithms, and dimensionality reduction algorithms; reinforcement learning models such as Q-learning and Deep Q Network; and deep learning models such as long short-term memory networks (LSTMs).

In some embodiments, the movement categorization module 131 and the machine learning module 132 may engage in a collaborative process prior to finalizing the movement categorization 140. Specifically, the movement categorization module 131 may initially process the digital signal 120 to form preliminary categorizations, which it then communicates to the machine learning module 132. The machine learning module 132, upon receiving these preliminary categorizations, may refine or adjust its own categorizations. Conversely, the machine learning module 132 may first generate its own preliminary categorizations and subsequently transmit these to the movement categorization module 131. The movement categorization module 131 may then perform a comparative analysis between its own initial categorizations and those received from the machine learning module 132. This iterative exchange of insights allows for a more nuanced and accurate generation of the final movement categorization 140, leveraging the strengths of both modules to enhance the overall interpretative accuracy.

The movement categorization 140 can include any number of predetermined actions of the user of wearable element 110, including without limitation: throwing and catching an object; grabbing; pulling; hitting; palm open or closed; hand balled into a fist; pointing a finger; motioning the hand in a particular way; jumping; kicking; walking; running; waving; clapping; typing; swimming; dancing; playing an instrument; performing martial arts; and performing yoga.

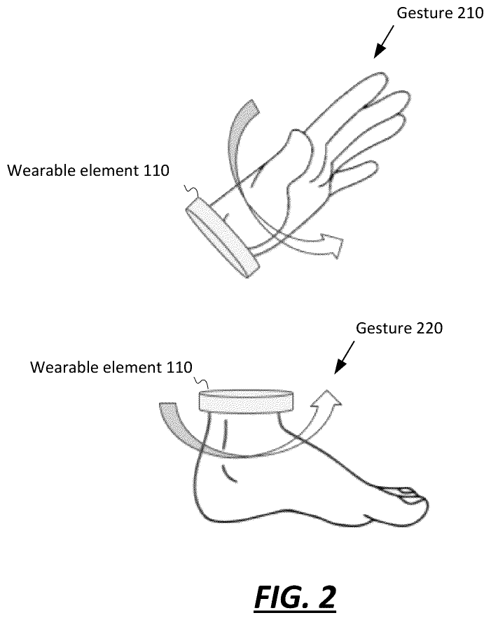

FIG. 2 illustrates wearable elements according to exemplary embodiments. As illustrated, the wearable element 110 may be worn around the wrist and/or ankle of the user. The wearable element 110 may measure the flexion of the muscles surrounding the body part. For example, in gesture 210 the wearable element is worn around wrist, the piezoelectric transducers 111 will generate an analog signal in response the force applied to the piezoelectric transducers 111 by the user moving his hand. As another example, in gesture 220 the wearable element 110 is worn around the ankle, the piezoelectric transducers 111 will generate an analog signal in response to the force applied by the user moving their foot. As previously stated, the wearable element 110 may be placed anywhere on the user's body and may take the form of a strap, patch, band, glove, wristband, headband, sleeve, or other item that can be worn or removably attached to a human user.

FIG. 3 is a block diagram illustrating circuitry 300 according to example embodiments. The circuitry 300 may include a transducer 305, a resistor 310, a diode 315, a resistor 320, and a microprocessor 325. The transducer 305 may be operable attached to one or more piezoelectric transducers 111. Each of the many piezoelectric transducers 111 may be associated with one or more transducers 305. Instead, the circuitry 300 may be powered by the electric signal produced by the piezoelectric transducers 111. The transducer 305 may generate one or more ultrasonic, analog signals corresponding to the energy released by the piezoelectric transducers 111 in response to the wearer's muscle flexion. The transducers 305 then generate an analog signal that is directly proportional to the applied pressure. The analog signal may pass through the resistor 310, diode 315, and resistor 320 before arriving at the microprocessor 325. The resistors 310 and 320 can include a passive two-terminal electrical components that implements electrical resistance. The diode 315 can include a two-terminal electronic component that conducts current primarily in one direction. In the context of this wearable device, resistors 310 and 320 are used to control the flow of electric current. They limit the amount of current passing through them to a level that is safe for the microprocessor 325 to handle. In some embodiments, this analog signal is generated through an analog process, which requires no input power, thus allowing the system to operate with zero power consumption from the sensors.

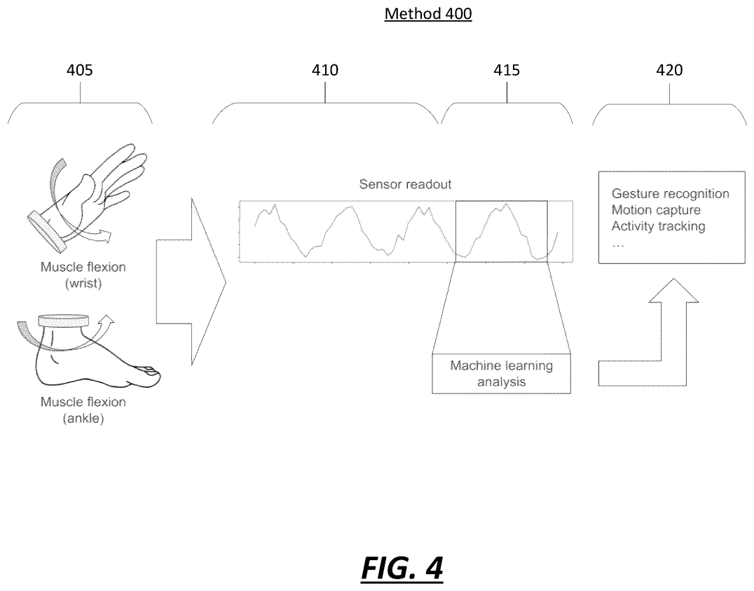

FIG. 4 is a diagram illustrating a method 400 of generating a movement recognition based on a digital signal produced by example embodiments. At step 405, the wearable elements 110 including the piezoelectric transducers 111 can generate analog signals in response to muscle flexion, send those analog signals to the ADC 113 via the circuitry 112, and convert those analog signals to digital signals 120. At step 410, those digital signals 120 can be sent to the movement interpretation processor 130. As illustrated in the figure, the digital signal 120 may include a graph or digital readout of the signal. At step 415, the machine learning module 132 of the movement interpretation processor 130 may interpret some or all of the digital signal 120 to determine what gesture is being performed. In some embodiments, step 415 may include recording the digital signal 120 as a time series. At step 420, the movement categorization module 131 may categorize the gesture, movement, or other activity being performed by the user based on the module's analysis of the time series of the digital signal 120. In other example embodiments, the machine learning module 132 may analyze the time series of the digital signal 120 and, based on this analysis, generate a movement categorization 140.

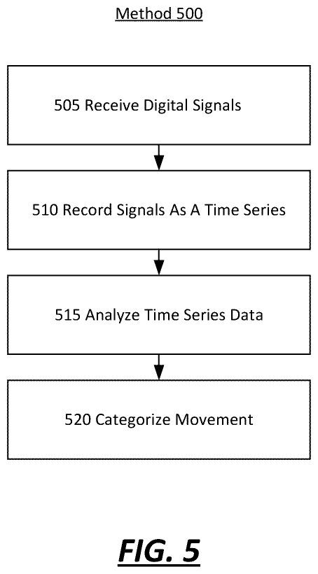

FIG. 5 is a flowchart illustrating a method 500 according to example embodiments. In the method 500, the movement interpretation processor 130 may determine what motion the wearer is performing based on the digital signals 120 from the piezoelectric transducers 111 on the wearable element 110.

At step 505, the movement interpretation processor 130 receive one or more digital signals 120. These digital signals 120 can be received continuously or in batches. At step 510, the processor records signals as a time series. The time series may be a sequence of numerical data points in successive order. In this context, each data point represents a digital signal from the piezoelectric transducers 111 at a specific point in time. At step 515, the processor analyzes the time series. This analysis can be performed by either or both the movement categorization module 131 and the machine learning module 132. In example embodiments, the machine learning module 132 may decompose the time series data into its constituent components, such as trend, seasonality, and noise. Then, the machine learning module 132 may extract features from the time series data that are indicative of specific movements or gestures. These features could include statistical measures such as mean, variance, and skewness, as well as more complex features like Fourier transforms or wavelet coefficients that capture the frequency content of the signal. In some embodiments, the movement categorization module 131 may determine, based on the digital signal 120, that there is significant muscle flexion around the muscles associated with the user closing their palm and making a fist. At step 520, the processor categorizes the movement based on the analysis of the time series. In some embodiments, the movement categorization module 131 may analyze the digital signal 120 and based on this analysis, determine which movement categorization 140 best fits the digital signal 120. Thus, the movement categorization module 131 may generate a movement categorization 140 of the user making a first gesture with their hand. In other embodiments, the machine learning module 132 may make the determination.

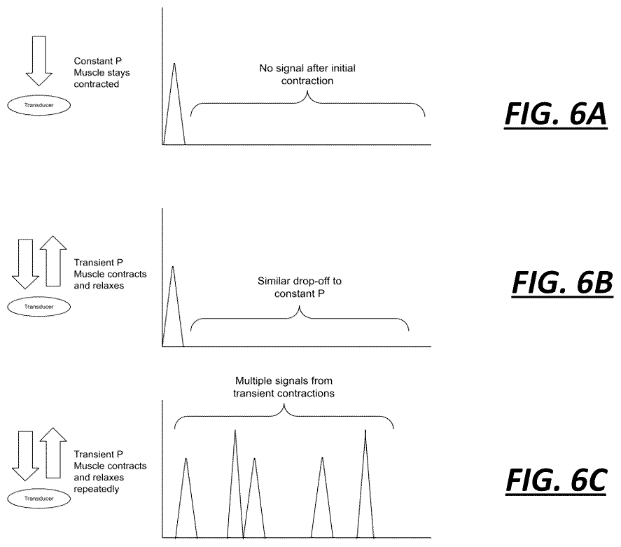

FIGS. 6A, 6B, and 6C illustrate the conventional graphical readout of piezoelectric transducers according to example embodiments. Generally, piezoelectric transducers 111 are highly sensitive to dynamic pressure events. That is, they produce a signal only when there is some change in force applied to them. However, when there is no pressure or constant pressure, the piezoelectric transducers 111 will produce no signal or a very weak signal. This leads to a significant problem when generating signals based on muscle flexion. Referring to FIG. 6A, when a muscle is contracted or flexed continuously, the transducer will produce a signal that is momentarily strong when the flexion occurs, but then the signal is flat when the muscle stays flexed. Referring to FIG. 6B, a similar result may occur when a muscle contracts then relaxes: the signal is strong when the muscle is momentarily flexed, but then the signal runs flat when the muscle relaxes. Although two different events are occurring in FIGS. 6A and 6B, the two-dimensional representation (time vs. sensor readout) of the transducer signal makes it difficult to distinguish between the two. Referring to FIG. 6C, when a muscle is repeatedly contracted and relaxed multiple times over a longer timescale, a single transducer will produce a signal that momentarily peaks during contraction and drops off in the intervals between contraction, similar to FIG. 6B. FIGS. 6A, 6B, and 6C demonstrate the efficacy of recording muscle activity at a single point, but this form of analysis may not provide full insight as to gross motor activation, for example, a gesture being performed by a user.

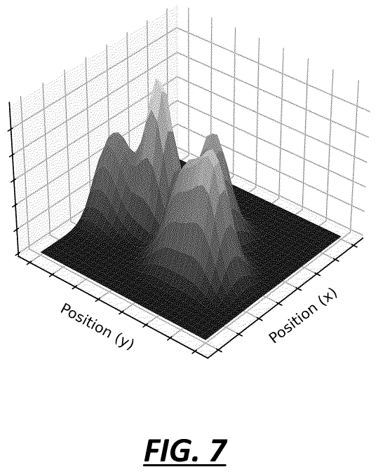

However, some embodiments may employ a combination of multiple transducer signals across different locations, to better understand the muscle activity generated by the wearer. FIG. 7 is a three-dimensional graph of multiple transducer signals obtained at a simultaneous time step, according to example embodiments. FIG. 7 illustrates what signals from multiple piezoelectric transducers 111 would look like in a three-dimensional space at a given moment in time as opposed to the two-dimensional space illustrated in FIGS. 6A and 6B. By plotting the pressure values on the Z-axis from each sensor along an X and Y plane (e.g., element 1 is at position (x1, y1), element 2 at (x2, y2), etc.), the system 100 can deduce more information about the wearer's movement than it could have by analyzing each signal independently.

By analyzing the digital signals in three dimensions, the movement interpretation processor 130 can discern the shape and contour of the muscle activity over time. The movement interpretation processor 130, via the movement categorization module 131 and the machine learning module 132, analyzes the three-dimensional data to identify patterns that correspond to specific movements or gestures. For example, a flexing motion might create a distinct arc shape in the three-dimensional space, while a twisting motion might generate a different shape. The movement interpretation processor 130 can compare these patterns against a database of known movement signatures to categorize the wearer's action. Furthermore, the temporal aspect of the movements, which is the change in pressure values over time, can be visualized as evolving shapes in the three-dimensional space. This allows the system to distinguish between static muscle contractions and dynamic movements, as well as to recognize complex gestures that involve multiple sequential muscle activations. In some embodiments, the machine learning module 132 can be trained to recognize a wide array of movements by analyzing the three-dimensional pressure data.

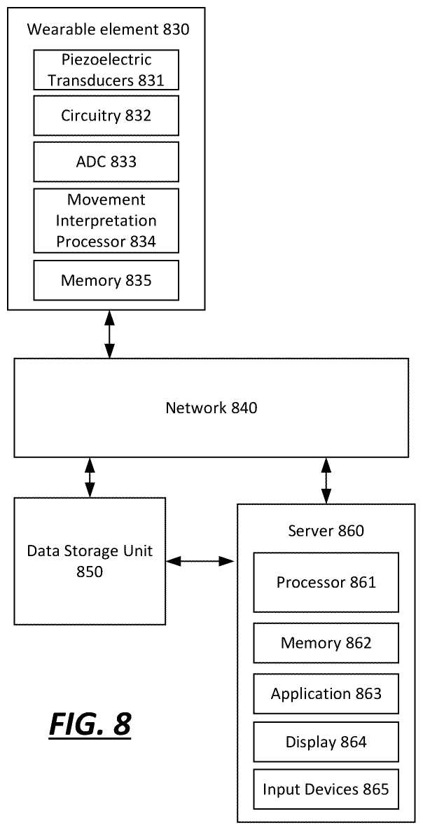

FIG. 8 illustrates a system 800 according to an example embodiment. The system 800 may include a wearable element 830, network 840, data storage unit 850, and a server 860. Although FIG. 8 illustrates single instances of components of system 800, system 800 may include any number of components.

The system 800 can include one or more wearable elements 830 including one or more piezoelectric transducers 831, circuitry 832, ADC 833, movement interpretation processor 834, and memory 835 with reference to FIG. 1. The wearable element 830 may be in communication with the data storage unit 850 and server 860 over the network 840. For example, wearable element 830 may store one or more data points associated with the digital signal 120 and movement categorizations 140. The memory 835 may be a read-only memory, write-once read-multiple memory or read/write memory, e.g., RAM, ROM, and EEPROM, and the server 860 may include one or more of these memories. A read-only memory may be factory programmable as read-only or one-time programmable. One-time programmability provides the opportunity to write once then read many times. A write-once read-multiple memory may be programmed at one point in time. Once the memory is programmed, it may not be rewritten, but it may be read many times. A read/write memory may be programmed and re-programed many times after leaving the factory. It may also be read many times. The memory 835 may be configured to store one or more software applications.

System 800 may include one or more networks 840. In some embodiments, the network 840 may be one or more of a wireless network, a wired network or any combination of wireless net-work and wired network and may be configured to connect the wearable elements 830, the server 860, and database or data storage unit 850. For example, the network 840 may include one or more of a fiber optics network, a passive optical network, a cable net-work, an Internet network, a satellite network, a wireless local area network (LAN), a Global System for Mobile Communication, a Personal Communication Service, a Personal Area Net-work, Wireless Application Protocol, Multimedia Messaging Service, Enhanced Messaging Service, Short Message Service, Time Division Multiplexing based systems, Code Division Multiple Access based systems, D-AMPS, Wi-Fi, Fixed Wireless Data, IEEE 802.11b, 802.15.1, 802.11n and 802.11g, Bluetooth, NFC, Radio Frequency Identification (RFID), Wi-Fi, and/or the like.

In addition, the network 840 may include, without limitation, telephone lines, fiber optics, IEEE Ethernet 902.3, a wide area network, a wireless personal area network, a LAN, or a global net-work such as the Internet. In addition, the network 840 may support an Internet network, a wire-less communication network, a cellular network, or the like, or any combination thereof. The network 840 may further include one network, or any number of the exemplary types of net-works mentioned above, operating as a stand-alone network or in cooperation with each other. The network 840 may utilize one or more protocols of one or more network elements to which they are communicatively coupled. The network 840 may translate to or from other protocols to one or more protocols of network devices. Although the network 840 is depicted as a single network, it should be appreciated that according to one or more examples, the network 840 may include a plurality of interconnected networks, such as, for example, the Internet, a service provider's network, a cable television network, corporate networks, such as credit card association networks, and home networks. The network 840 may further include, or be configured to create, one or more front channels, which may be publicly accessible and through which communications may be observable, and one or more secured back channels, which may not be publicly accessible and through which communications may not be observable.

System 800 may include a database or data storage unit 850. The database or data storage unit 850 may include a relational database, a non-relational database, or other database implementations, and any combination thereof, including a plurality of relational databases and non-relational databases. In some examples, the database or data storage unit 850 may include a desktop database, a mobile database, or an in-memory database. Further, the database or data storage unit 850 may be hosted internally by the server 860 or may be hosted externally of the server 860, such as by a server, by a cloud-based platform, or in any storage device that is in data communication with the server 860.

The system can include a server 860. The server 860 may be a network-enabled computer de-vice. Exemplary network-enabled computer devices include, without limitation, a server, a net-work appliance, a personal computer, a workstation, a phone, a handheld personal computer, a personal digital assistant, a thin client, a fat client, an Internet browser, a mobile device, a kiosk, a contactless card, or other a computer device or communications device. For example, network-enabled computer devices may include an iPhone, iPod, iPad from Apple® or any other mobile device running Apple's iOS® operating system, any device running Microsoft's Windows® Mobile operating system, any device running Google's Android® operating system, and/or any other smartphone, tablet, or like wearable mobile device. The server may be a com-bination of one or more cloud computing systems such as public clouds, private clouds, and hybrid clouds.

The server 860 may include a processor 861, a memory 862, and an application 863. The processor 861 may be a processor, a microprocessor, or other processor, and the server 860 may include one or more of these processors. The processor 861 may include processing circuitry, which may contain additional components, including additional processors, memories, error and parity/CRC checkers, data encoders, anti-collision algorithms, controllers, command decoders, security primitives and tamper-proofing hardware, as necessary to perform the functions described herein.

The processor 861 may be coupled to the memory 862. The memory 862 may be a read-only memory, write-once read-multiple memory or read/write memory, e.g., RAM, ROM, and EEPROM, and the server 860 may include one or more of these memories. A read-only memory may be factory programmable as read-only or one-time programmable. One-time programmability provides the opportunity to write once then read many times. A write-once read-multiple memory may be programmed at one point in time. Once the memory is programmed, it may not be rewritten, but it may be read many times. A read/write memory may be programmed and re-programed many times after leaving the factory. It may also be read many times. The memory 862 may be configured to store one or more software applications, such as the application 863.

The application 863 may include one or more software applications, such as a mobile application and a web browser, including instructions for execution on the server 860. In some examples, the server 860 may execute one or more applications, such as software applications, that enable, for example, network communications with one or more components of the system 800, transmit and/or receive data, and perform the functions described herein. Upon execution by the processor 861, the application 863 may provide the functions described in this specification, specifically to execute and perform the steps and functions in the process flows described below. Such processes may be implemented in software, such as software modules, for execution by computers or other machines. The application 863 may provide graphical user interfaces (GUIs) through which a user may view and interact with other components and devices within the system 800. The GUIs may be formatted, for example, as web pages in Hyper-Text Markup Language (HTML), Extensible Markup Language (XML) or in any other suitable form for presentation on a display device depending upon applications used by users to interact with the system 800.

The server 860 may be associated with one or more of a display 864 or input devices 865. The display 864 may be any type of device for presenting visual information such as a computer monitor, a flat panel display, and a mobile device screen, including liquid crystal displays, light-emitting diode displays, plasma panels, and cathode ray tube displays. The input devices 865 may include any device for entering information into the server 860 that is available and supported by the wearable elements 830, such as a touchscreen, keyboard, mouse, cursor-control device, touchscreen, microphone, digital camera, video recorder or camcorder. These devices may be used to enter information and interact with the software and other devices described herein.

### Applications

The wearable element described in the patent application has profound implications for the field of virtual and extended reality. By capturing muscle activity with high precision, the device can translate even the subtlest of human movements into digital input, enhancing the immersive experience in VR/XR environments. Users can interact with virtual objects and interfaces with natural gestures and movements, making the virtual experience more intuitive and engaging. This technology can be particularly transformative in gaming, education, and professional training simulations, where accurate motion tracking is paramount for a realistic experience. The ability to employ a high density of sensors without the associated power costs allows for a more detailed and accurate capture of muscle activity. This leads to a more precise translation of human movements into digital input, thereby improving the immersive experience in VR/XR environments.

In the realm of athletics, the wearable element serves as a sophisticated tool for performance analysis and enhancement. By monitoring muscle activity during training and competition, coaches and athletes can gain insights into biomechanics, muscle engagement, and movement efficiency. This data can be used to tailor training programs, reduce the risk of injury, and optimize performance. For instance, a runner's gait can be analyzed to correct posture and stride, while a swimmer's stroke can be refined for maximum propulsion. The ability to capture data without the encumbrance of heavy equipment or the distraction of external sensors is a game-changer for sports analytics. In athletics, the high sensor density enables a more comprehensive analysis of muscle engagement and movement efficiency, contributing to optimized performance.

The wearable's ability to monitor and categorize muscle activity has direct applications in physical therapy and rehabilitation. Therapists can use the device to track patients' progress in real-time, ensuring that exercises are performed correctly and that the targeted muscles are being activated. This can be particularly beneficial for stroke recovery, where retraining muscles is a gradual process that requires precise, consistent feedback. The device's sensitivity to pressure differentials allows for the detection of even minor improvements in muscle control, which can be encouraging for patients and informative for therapists.

For individuals with Alzheimer's and other cognitive disabilities, the wearable element can provide support by monitoring daily activities and ensuring safety. The device's gesture recognition capabilities can help in identifying patterns that may indicate confusion or distress, allowing caregivers to intervene promptly. Additionally, for those with motor disabilities, the device can facilitate communication through gesture-based interfaces, empowering users to interact with their environment and maintain independence. Furthermore, the lack of a power source for the sensors eliminates the risk of power failure, ensuring reliable and continuous monitoring of muscle activity. This is particularly beneficial in health monitoring, where consistent data collection is paramount. For individuals with Alzheimer's and other cognitive disabilities, the device's ability to provide continuous, real-time data without the risk of power failure ensures safety. For those with motor disabilities, the device's reliable gesture recognition capabilities facilitate communication through gesture-based interfaces.

Beyond these specific applications, the wearable element's technology holds promise for broader health monitoring and assistance for various disabilities. By providing continuous, real-time data on muscle activity, it can serve as an early warning system for the onset of musculoskeletal conditions or the exacerbation of existing ones. For individuals with disabilities, the technology can be integrated into assistive devices, enhancing their functionality and responsiveness to the user's intent, thereby improving quality of life and autonomy.

While the foregoing is directed to embodiments described herein, other and further embodiments may be devised without departing from the basic scope thereof. For example, aspects of the present disclosure may be implemented in hardware or software or a combination of hard-ware and software. One embodiment described herein may be implemented as a program product for use with a computer system. The program(s) of the program product define functions of the embodiments (including the methods described herein) and may be contained on a variety of computer-readable storage media. Illustrative computer-readable storage media include, but are not limited to: (i) non-writable storage media (e.g., read-only memory (ROM) devices within a computer, such as CD-ROM disks readably by a CD-ROM drive, flash memory, ROM chips, or any type of solid-state non-volatile memory) on which information is permanently stored; and (ii) writable storage media (e.g., floppy disks within a diskette drive or hard-disk drive or any type of solid state random-access memory) on which alterable information is stored. Such computer-readable storage media, when carrying computer-readable instructions that direct the functions of the disclosed embodiments, are embodiments of the present dis-closure.

It will be appreciated to those skilled in the art that the preceding examples are exemplary and not limiting. It is intended that all permutations, enhancements, equivalents, and improvements thereto are apparent to those skilled in the art upon a reading of the specification and a study of the drawings are included within the true spirit and scope of the present disclosure. It is therefore intended that the following appended claims include all such modifications, permutations, and equivalents as fall within the true spirit and scope of these teachings.

What is claimed is:

1. A system for capturing muscle activity, comprising:
a wearable device comprising:
one or more piezoelectric elements configured to convert one or more mechanical deformations associated with muscle activity into an analog signal;
an analog-to-digital converter (ADC) electrically coupled to the piezoelectric elements, the ADC being configured to convert the analog signal into a digital signal; and
a processor in communication with the ADC, wherein the processor is configured to receive and analyze the digital signal,
wherein the processor is further configured to determine, based on the analysis, an action associated with the digital signal based on the analysis.

2. The system of claim 1, wherein the wearable device is integrated into a piece of clothing.

3. The system of claim 1, wherein the piezoelectric elements are placed at points of muscular activity on one or more arms and legs of a user wearing the wearable device.

4. The system of claim 1, wherein the processor comprises a machine learning model trained to recognize patterns in one or more time series data of the digital signal that correspond to specific movements, gestures, or activities.

5. The system of claim 4, wherein the machine learning model is configured to differentiate between walking, running, and jumping based on the patterns of pressure differentials associated with a wearer.

6. The system of claim 1, wherein the processor operates with zero power consumption from the piezoelectric elements.

7. The system of claim 1, wherein the processor is further configured to determine an action based one or more stored digital signals corresponding with one or more predetermined actions.

8. A method for capturing muscle activity, comprising:
providing a wearable device including one or more piezoelectric elements;
positioning the one or more piezoelectric elements in proximity to a user's muscles;
converting, by the piezoelectric elements, mechanical deformations associated with muscle activity into an analog signal;
converting, by an analog-to-digital converter (ADC) electrically coupled to the piezoelectric elements, the analog signal into a digital signal;
analyzing, by a processor in communication with the ADC, the digital signal; and
determining, by the processor, an action associated with the digital signal based on the analysis.

9. The method of claim 8, wherein the wearable device is integrated into a piece of clothing.

10. The method of claim 8, wherein the piezoelectric elements are placed at points of muscular activity on one or more arms and legs of a wearer.

11. The method of claim 8 further comprising recognizing, by a machine learning model, one or more patterns in the digital signal that correspond to specific movements, gestures, or activities.

12. The method of claim 11 further comprising differentiating, by the machine learning model, between walking, running, and jumping based on the patterns of pressure differentials associated with these activities.

13. The method of claim 8 wherein the processor operates with zero power consumption from the piezoelectric elements.

14. The method of claim 8, wherein the wearable device is configured to be worn on various and multiple parts of a body.

15. The method of claim 8 further comprising recording, by the processor, the digital signal spatially as a time series.

16. The method of claim 15, wherein the piezoelectric elements are integrated into a fabric of the wearable device.

17. The method of claim 16, wherein the fabric is a part of a garment selected from the group consisting of: a shirt, a pair of pants, a wristband, and a headband.

18. The method of claim 15, wherein the piezoelectric elements are arranged in a pattern corresponding to a specific muscle group on a human body.

19. The method of claim 15, wherein the processor is configured to perform a time series analysis on the digital signal to identify patterns corresponding to specific movements, gestures, or activities.

20. A non-transitory computer readable medium containing computer executable instructions that, when executed by a computer hardware arrangement, cause the computer hardware arrangement to perform procedures comprising:
converting mechanical deformations associated with muscle activity into an analog signal;
converting the analog signal into a digital signal;
analyzing, by a machine learning algorithm, the digital signal; and
determining an action associated with the digital signal based on the analysis.
